(19) United States
(12) Patent Application Publication
(10) Pub. No.: US 2006/0101882 A1
(43) Pub. Date: May 18, 2006

(54) Use of animal urine for efficient and quality vermicomposting and recycling slow degrading and unconventional substrates and the process for the same

(75) Inventors: Suman Preet Singh Khanuja, Lucknow (IN); Alok Kalra, Lucknow (IN); Ranganathan Santha Kumar Tiruppadiripuliyur, Lucknow (IN); Mahendra Pandurang Darokar, Lucknow (IN); Ajit Kumar Shasany, Lucknow (IN); Dharni Dhar Patra, Lucknow (IN); Virendra Kumar Singh Tomar, Lucknow (IN); Om Parkash Dhawan, Lucknow (IN); Rakesh Pandey, Lucknow (IN); Ravi Prakash Bansal, Lucknow (IN); Raj Kishori Lal, Lucknow (IN); Govind Ram, Lucknow (IN); Anirban Pal, Lucknow (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/331,973

(22) Filed: Jan. 13, 2006

Related U.S. Application Data
(62) Division of application No. 10/403,816, filed on Mar. 31, 2003.

(51) Int. Cl. C05F 3/00; C12N 3/00

(52) U.S. Cl. 71/22; 435/242

(57) ABSTRACT

The present invention relates to an efficient process of vermicomposting and production of high-quality vermicompost from agro-waste(including distillation waste) using animal urine such as cattle urine.

### FIELD OF THE INVENTION

The present invention relates to an efficient process of vermicomposting and production of high-quality vermicompost from agro-waste(including distillation waste) using animal urine such as cattle urine.

### BACKGROUND OF THE INVENTION

It is well known that most of the biodegradable organic wastes can be converted in to vermicompost. (Singh and Rai, 1998, Yojna 10-12). Earlier an efficient vermicultivation process for converting distillation waste into quality vermicompost with the major advantages of faster composting process and superior quality-vermicompost (U.S. Pat. No. 6,488,733) has been developed. In India, among cattle cow is considered as a sacred animal whose urine is traditionally considered useful. Apart from its conventional use in manures it is even used in traditional medicine preparations. Its possible role in pharmaceutical industries as bioenhancer was established recently in our laboratory (U.S. Pat. No. 6,410,059). Cattle urine is logically a nitrogen source and has been used as manures/fertilizer source (Sharma, 2001, A hanbook of organic farming, Agrobios India) to supplement the nutritional requirement of the crop. The object of the present invention is to improve the efficiency/rapidity of the vermicomposting process and quality of the vermicompost produced by the use of cattle's urine. It is also the object of the present invention to develop a process for recycling of those plant wastes as substrates, which are either tough/fiber rich, or is not preferred by earthworms.

### OBJECTS OF THE INVENTION

The main object of the invention is to provide a composting composition which enables the use of animal urine to recycle slow degrading and unconventional substrates such as some agricultural wastes.

It is another object of the invention to provide a vermicomposting composition using animal urine such as cattle urine which shows higher nutritional value.

### SUMMARY OF THE INVENTION

Accordingly the present invention provides a composting composition comprising a mixture of 5 to 90% of animal urine mixed in of chopped agricultural waste.

In one embodiment of the invention, the composting composition is a vermicomposting composition.

In another embodiment of the invention, the earthworms are selected from Perionyx excavatus, Eisenia fetida, Eudrilus eugeniae and Eisenia andrie.

In another embodiment of the invention, the chopped agricultural waste comprises distilled waste of the crops selected from the group consisting of scented geranium (Pelargonium graveolens), citronella (C. winterianus), menthol mint (Mentha arvensis), agro-waste from rice straw, Brassica waste and sugarcane waste.

In another embodiment of the invention, the agricultural waste comprises non-conventional agro-waste which are not easily decomposed selected from pine needles and sugarcane trash.

In another embodiment of the invention, the non-conventional agro-waste is ordinarily not preferred as such by worms for their growth.

In another embodiment of the invention, the agricultural waste comprise waste from medicinal/spice crop/plants selected from group consisting of waste from Plantago ovata, Papavar someniferum, Coriandrum sativum and Foeniculum vulgarae.

In yet another embodiment of the invention, the animal urine is selected from urine of cattle species and domestic animals.

In yet another embodiment of the invention, the animal urine is urine of buffalo, horse, pigs, sheep and goat.

In yet another embodiment of the invention, the agricultural waste is selected from wheat straw and distillation waste of lemongrass (Cymbopogon flexuosus).

In another embodiment of the invention, the compost has a nutritional status of N in the range of 1.32 to 1.60%; P in the range of 0.56 to 0.71; K in an amount of 391 to 574 ppm; Fe in an amount of 340 to 706 ppm; Zn in an amount of 260 to 340 ppm; Mn in an amount of 256 to 305 ppm, organic carbon in a concentration of 13.0 to 15.6%; and a pH in the range of 6.06 to 6.94.

In another embodiment of the invention, the waste material comprises chopped distillation waste of lemongrass and the population of microbes therein after application of animal urine is bacteria of about 3.8×10⁵; fungi of about 2.6×10⁵; and actinomycetes of about 2.9×10⁵.

A method for vermicomposting comprising (a) drying and chopping of agricultural waste; (b) treatment of the dried and chopped agricultural waste with animal urine in a concentration of 5% to 90%; (c) transferring the treated chopped agricultural waste to containers containing earthworms and maintaining the moistness thereof using water; (d) harvesting the compost and separating the earthworms therefrom.

In another embodiment of the invention, the earthworms are selected from Perionyx excavatus, Eisenia fetida, Eudrilus eugeniae and Eisenia andrie.

In another embodiment of the invention, the chopped agricultural waste comprises distilled waste of the crops selected from the group consisting of scented geranium (Pelargonium graveolens), citronella (C. winterianus), menthol mint (Mentha arvensis), agro-waste from rice straw, Brassica waste and sugarcane waste.

In another embodiment of the invention, the agricultural waste comprises non-conventional agro-waste which are not easily decomposed selected from pine needles and sugarcane trash.

In another embodiment of the invention, the non-conventional agro-waste is ordinarily not preferred as such by worms for their growth.

In another embodiment of the invention, the agricultural waste comprise waste from medicinal/spice crop/plants selected from the group consisting of waste from Plantago ovata, Papavar someniferum, Coriandrum sativum and Foeniculum vulgarae.

In yet another embodiment of the invention, the animal urine is selected from urine of cattle species and domestic animals.

In yet another embodiment of the invention, the animal urine is urine of buffalo, cow, horse, pig, sheep or goat.

In yet another embodiment of the invention, the agricultural waste is selected from wheat straw and distillation waste of lemongrass (Cymbopogon flexuosus).

In yet another embodiment of the invention, the waste comprises distillation waste obtained after distillation of lemongrass herbage for 3 h at 20-25 lbs. steam inlet pipe pressure for 72 h and wheat straw obtained 2 months after harvest of wheat and pine needles, which are chopped into small pieces of size 10-15 cm.

In yet another embodiment of the invention, the treated agricultural waste is transferred to a container containing earthworms (Perionyx excavatus) and dry unrotten cattle dung.

In yet another embodiment of the invention, the harvesting of the compost is carried out after about 90 days and then dried for about 72 h and the dried material sieved to separate the compost the earthworms and undecomposed material.

In another embodiment of the invention, the compost has a nutritional status of N in the range of 1.32 to 1.60%; P in the range of 0.56 to 0.71; K in an amount of 391 to 574 ppm; Fe in an amount of 340 to 706 ppm; Zn in an amount of 260 to 340 ppm; Mn in an amount of 256 to 305 ppm, organic carbon in a concentration of 13.0 to 15.6%; and a pH in the range of 6.06 to 6.94.

In another embodiment of the invention, the waste material comprises chopped distillation waste of lemongrass and the population of microbes therein after application of animal urine is bacteria of about 3.8×10⁵; fungi of about 2.6×10⁵; and actinomycetes of about 2.9×10⁵.

### DETAILED DESCRIPTION OF THE INVENTION

Experiments have been carried out and a process has been developed wherein addition of cattle's urine results in faster multiplication of worms, shorter composting period, accelerated pace of composting in slow-degrading substrates and high-quality vermicompost from the wheat straw and distillation waste of lemongrass (Cymbopogon flexuosus).

The process comprises of a) drying of distillation waste (obtained after distillation of lemongrass herbage for 3 h at 20-25 lbs. steam inlet pipe pressure) for 72 h; b) chopping of distillation waste and wheat straw (2 months after harvest of wheat) and pine needles a slow-degrading wastes into small pieces (−10-15 cm); c) treatment with different concentrations of cattle's urine as in Table 1, d) transferring 500 g of chopped treated material into cement pots (15″×15″×15″) containing about 3″ layer(200 g) of unrotten cowdung and fifty earthworms (Perionyx excavatus); e) daily watering of the pots to keep the plant material moist; f) harvesting of compost after 90 days and drying for 72 h; g) sieving of the dried material to separate earthworms and the undecomposed material

The invention is described in detail in the examples given below which are illustrative and therefore should not be construed as limiting the scope of the present invention.

### EXAMPLE 1

A worm bed of 4.5 m long, 1.2 m wide and 0.5 m deep was prepared in a pit. The base of the pit was covered with coarse sand (4-5 cm layer) followed by another layer of field soil (4-5 cm) and a layer of partially decomposed cowdung (7-10 cm). The inner sides of the pit were lined with bricks (22×11×7 cm). The worms Perionyx excavatus 2000/pit were released into the beds. The dried rice straw chopped into small pieces mixed with unrotten cow dung was placed in the beds, filling the beds to the top. The beds were watered daily and were kept moist. These were turned frequently (atleast twice a week) for proper aeration. After 4 months the well-grown worms (8-10 cm long) were used for composting.

### EXAMPLE II

An experiment was conducted to study the effect of cattle's urine on cultivation/multiplication of worms and time to compost. The experiment was conducted in cement pots(15′×15′×15′) in which a base layer of partially rotten cowdung(200 g) was provided. Fifty worms were added and the pots were loaded with 500 g of agro/distillation waste. Observation regarding the total number of earthworms was recorded 90 days after loading of waste. The harvested compost was sieved to separate undecomposed material and the resultant sieved compost produced was taken as indicator of faster composting process. It was found that the distillation waste of Cymbopogon flexuosus is better substrates for multiplication of earthworms. The number of worms harvested after 90 days were in general higher in the pots treated with urine and the increase was marked in wheat straw. As found earlier distillation wastes are better substrates for vermicomposting, the amount of compost produced was much more than the compost harvested from wheat straw. Addition of cattle's urine improved the efficiency of the composting process as the harvested weight of vermicompost produced from distillation waste of lemongrass and wheat straw was significantly higher from cattle's urine treated pots (Table 1).

TABLE 1
Effects of application of cattle's
urine(CU) on vermicomposting process

| | | Weight(g) of |
|---|---|---|
| | No. of | compost(shade |
| Treatment | worms | dried for 72 h) |
| Wheat Straw + CU (50%) | 210 | 343 |
| Wheat Straw + CU (25%) | 165 | 301 |
| Wheat Straw + CU (10%) | 178 | 244 |
| Wheat Straw + CU (5%)* | 210 | 337 |
| Wheat Straw | 181 | 204 |
| Lemongrass distillation waste + CU (50%) | 229 | 531 |
| Lemongrass distillation waste + CU (25%) | 207 | 478 |
| Lemongrass distillation waste + CU (10%) | 206 | 416 |
| Lemongrass distillation waste + CU (5%)* | 210 | 498 |
| Lemongrass distillation waste | 203 | 407 |

*Sprayed fortnightly (@50 ml/pot), all others treated for 30 min. before adding the material into pots

### EXAMPLE III

Some plant wastes are not easily biodegradable/compostable probably because of high lignin/silica content. Earthworms also do not directly ingest these unless these are decomposed to some extent. A preliminary experiment was conducted to test the usefulness of cattle's urine in enhancing efficiency of vermicomposting in materials, which are difficult to compost such as pine needles. The results of the experiments showed that addition of cattle's urine (5% urine sprayed fortnightly @500 ml/pot) which further improved with the addition of fresh cowdung (500 g/pot applied in layers over the substrate). Presence of many enzymes and chemicals in cattle's urine/dung probably paced the digestion/degradation of such material or even attracted worms which might have been liked/easily ingested by earthworms

### EXAMPLE IV

The compost produced was air-dried and estimation concerning pH, total organic carbon, content of available nitrogen, phosphorus and potassium and micronutrients was carried out. Addition of cattle's urine reduced the pH values of the composts from both conventional as well as distillation waste to some extent. Nutritional quality of the composts produced from both the wastes was found to be superior with respect to nitrogen, phosphorus, potassium and some essential micronutrients if treated with cattle's urine (Table 2).

TABLE 2
Effect of application of cattle's urine on nutritional status of the compost

| | | | Org. | | | | | | |
|---|---|---|---|---|---|---|---|---|---|
| S. | | | Carbon | | | K | Fe | Zn | Mn |
| No | Treatment | pH | (%) | N(%) | P(%) | (ppm) | (ppm) | (ppm) | (ppm) |
| 1 | Wheat Straw + CU (50%) | 6.54 | 14.5 | 1.32 | 0.70 | 574 | 696 | 320 | 301 |
| 2 | Wheat Straw + CU (25%) | 6.94 | 13.3 | 1.39 | 0.59 | 542 | 340 | 286 | 305 |
| 3 | Wheat Straw + CU (10%) | 6.63 | 13.0 | 1.45 | 0.58 | 466 | 517 | 280 | 300 |
| 4 | Wheat Straw + CU (5%)* | 6.57 | 14.3 | 1.39 | 0.56 | 424 | 475 | 346 | 275 |
| 5 | Wheat Straw | 7.35 | 12.5 | 1.23 | 0.55 | 418 | 288 | 280 | 272 |
| 6 | Lemongrass distillation | 6.13 | 14.5 | 1.41 | 0.70 | 391 | 706 | 333 | 273 |
| | waste + CU (50%) | | | | | | | | |
| 7 | Lemongrass distillation | 6.06 | 13.8 | 1.60 | 0.63 | 421 | 653 | 340 | 269 |
| | waste + CU (25%) | | | | | | | | |
| 8 | Lemongrass distillation | 6.06 | 15.6 | 1.57 | 0.66 | 448 | 896 | 260 | 256 |
| | waste + CU (10%) | | | | | | | | |
| 9 | Lemongrass distillation | 6.11 | 13.3 | 1.48 | 0.71 | 466 | 687 | 340 | 262 |
| | waste + CU (5%)* | | | | | | | | |
| 10 | Lemongrass distillation waste | 6.39 | 11.9 | 1.11 | 0.44 | 426 | 412 | 300 | 162 |

### EXAMPLE V

The compost produced from distillation waste of lemongrass was also analysed for the microbial populations. The population of microbes (fungi, bacteria and actinomycetes) was higher in compost produced by using cattle's urine. The population of actinomycetes was markedly increased (Table 3).

TABLE 3
Effect of application of cattle's urine(CU) on population of microbes(/g)
in compost produced from distillation waste of lemongrass

| | | Vermicom- |
|---|---|---|
| Observation | vermicompost | post + CU |
| Fungi | 1.8 × 10⁵ | 2.6 × 10⁵ |
| Bacteria | 3.4 × 10⁵ | 3.8 × 10⁵ |
| Actinomycetes | 1.6 × 10⁵ | 2.9 × 10⁵ |

Vermicompost + CU = vermicomposting with cattle's urine

### EXAMPLE VI

The compost produced was also tested their influence on plant growth. The compost produced was tested on Lucerne (Medicago sativa) for improved plant growth. All the treatment receiving cattle's urine were clubbed together and compared with control(no urine). It was observed that application (10 t/ha) compost prepared using cattle's urine improved the plant height and herb yield (recorded 60 days after sowing) (Table 4).

TABLE 4
Effect of application of vermicompost on growth
and yield of Lucerne(Medicago sativa)

| Observation | vermicompost | Vermicompost + CU |
|---|---|---|
| Plant height(cm) | 34 | 41 |
| Plant weight(g/pot)* | 16 | 19 |

*average of 10 plants in a pot
Vermicompost + CU = vermicomposting with cattle's urine

Advantages
a) Superior quality compost rich in organic carbon, nitrogen phosphorus and potassium is produced by using cattle's urine from the distillation as well as other agro-wastes.
b) There is a faster multiplication of worms if distillation as well as other agro-wastes used are treated with cattle's urine.
c) Hard to compost materials can be composted at accelerated pace
d) Waste materials as such not preferred by earthworms can be converted into transformed substrates to be utilized by earthworms for composting
e) The compost produced after using cattle's urine contains higher concentration of nutrients than untreated one(no urine used)
f) The compost produced by using cattle's urine was rich in microbes particularly actinomycetes and such compost may be quite useful in combating plant diseases.
g) Vermicompost produced by the use of cattle's urine improved the growth of plant when compared with compost produced without cattle's urine. This indicate better performance of such composts in the field

1-12. (canceled)

13. A method for vermicomposting comprising (a) drying and chopping of agricultural waste; (b) treatment of the dried and chopped agricultural waste with animal urine in a concentration of 5% to 90%; (c) transferring the treated chopped agricultural waste to containers containing earthworms and maintaining the moistness thereof using water; (d) harvesting the compost and separating the earthworms therefrom.

14. A method as claimed in claim 13, wherein the earthworms are selected from Perionyx excavatus, Eiseniafetida, Eudrilus eugeniae and Eisenia andrie.

15. A method as claimed in claim 13, wherein the chopped agricultural waste comprises distilled waste of the crops selected from the group consisting of scented geranium (Pelargonium graveolens), citronella (C winterianus), menthol mint (Mentha arvensis), agro-waste from rice straw, Brassica waste and sugarcane waste.

16. A method as claimed in claim 13, wherein the agricultural waste comprises non conventional agro-waste which are not easily decomposed selected from pine needles and sugarcane trash.

17. A method as claimed in claim 16, wherein the non-conventional agro-waste is ordinarily not preferred as such by worms for their growth.

18. A method as claimed in claim 13, wherein the agricultural waste comprise waste from medicinal/spice crop/plants selected from the group consisting of waste from Plantago ovata, Papavar someniferum, Coriandrum sativum and Foeniculum vulgarae.

19. A method as claimed in claim 13, wherein the animal urine is selected from urine of cattle species and domestic animals.

20. A method as claimed in claim 13, wherein the animal urine is urine of buffalo, cow, horse, pig, sheep or goat.

21. A method as claimed in claim 13, wherein the agricultural waste is selected from wheat straw and distillation waste of lemongrass (Cymbopogon flexuosus).

22. A method as claimed in claim 13, wherein the waste comprises distillation waste obtained after distillation of lemongrass herbage for 3 h at 20-25 lbs. steam inlet pipe pressure for 72 h and wheat straw obtained 2 months after harvest of wheat and pine needles, which are chopped into small pieces of size 10-15 cm.

23. A method as claimed in claim 13, wherein the treated agricultural waste is transferred to a container containing earthworms (Perionyx excavatus) and dry unrotten cattle dung.

24. A method as claimed in claim 13, wherein the harvesting of the compost is carried out after about 90 days and then dried for about 72 h and the dried material sieved to separate the compost the earthworms and undecomposed material.

25. A method as claimed in claim 13, wherein the compost has a nutritional status of N in the range of 1.32 to 1.60%; P in the range of 0.56 to 0.71; K in an amount of 391 to 574 ppm; Fe in an amount of 340 to 706 ppm; Zn in an amount of 260 to 340 ppm; Mn in an amount of 256 to 305 ppm, organic carbon in a concentration of 13.0 to 15.6%; and a pH in the range of 6.06 to 6.94.

26. A method as claimed in claim 13, wherein the waste material comprises chopped distillation waste of lemongrass and the population of microbes therein after application of animal urine is bacteria of about 3.8×10⁵; fungi of about 2.6×10⁵; and actinomycetes of about 2.9×10⁵.
